(19) United States
(12) Patent Application Publication
(10) Pub. No.: US 2020/0129328 A1
(43) Pub. Date: Apr. 30, 2020

(54) BODY TEMPERATURE MANAGEMENT DEVICES AND METHODS

(71) Applicants: Bingren HU, Baltimore, MD (US); Chunli LIU, Baltimore, MD (US)

(72) Inventors: Bingren HU, Baltimore, MD (US); Chunli LIU, Baltimore, MD (US)

(21) Appl. No.: 16/493,777

(22) PCT Filed: Mar. 15, 2018
(86) PCT No.: PCT/US18/22666

§ 371 Date: Sep. 13, 2019

Related U.S. Application Data
(60) Provisional application No. 62/471,673, filed on Mar. 15, 2017.

(51) Int. Cl. A61F 7/12

(52) U.S. Cl. CPC A61F 7/123; A61F 2007/0054; A61F 2007/126

(57) ABSTRACT

Disclosed are devices and methods for body temperature management using a heat exchange device carrying a heat transfer medium to heat or cool a desired, focused portion of a patient's body. In accordance with certain aspects of the invention, the heat exchange device includes a steerable tip that may be manipulated via a steerable control apparatus to route the device through the patient's body to the intended location of treatment. Methods of using such heat exchange device to effect temperature management of the patient's organs are also disclosed.

### FIELD OF THE INVENTION

The present invention relates generally to the field of medicine, and more particularly to a method and apparatus for controlling and managing the temperature of humans and animals.

### BACKGROUND

The medical community has come to accept that induced therapeutic hypothermia may be used to achieve cardio-protection and neuro-protection. Currently there is sufficient evidence to regard therapeutic hypothermia as standard of care for situations such as patients with postanoxic encephalopathy. Further, therapeutic hypothermia has shown promising results in the treatment of head injury, stroke, subarachnoid hemorrhage (SAH), and in other situations. During emergency care and during surgery, inducing the hypothermic state may help to reduce local and systemic inflammation, tissue swelling, blood loss, and other adverse bodily reactions to hypoxia, ischemia, trauma, and surgical procedures. Medical professionals expect that other therapeutic benefits of inducing hypothermia to regulate body temperature will be discovered according to further development in this field.

There is still a need for more work and study to be done in order to identify the specific clinical situations in which hypothermia can be effective. However, many medical professionals have shifted their focus from assessment of clinical efficacy studies to finding ways to technically implement induced therapeutic hypothermia.

Currently there are three well known and used cooling techniques and systems: conventional cooling systems; surface cooling systems; and intravascular cooling systems. In addition to these cooling techniques, in recent years, a number of medical professionals and scientists have proposed cooling techniques via colorectal heat exchangers. For example, U.S. Pat. No. 6,641,602, by David P. Balding, the specification of which is incorporated herein by reference in its entirety, discloses a “Method and device including a colorectal heat exchanger.” The device disclosed in this patent includes a balloon-type heat exchange tube, which is used to regulate the temperature of a patient by inserting the heat exchange tube into the colon of the patient. The temperature of the heat exchange tube can be adjusted and/or controlled by running a cooling fluid through the tube. The heat exchange tube is inflated with a heat exchange fluid flowing into the tube from a temperature control unit. The fluid remains inside the tube and is not infused into the patient. The heat exchange tube includes therein an inflow lumen, an outflow lumen, and an irrigation lumen for irrigating the colon.

The cooling technique presented in U.S. Pat. No. 6,641,602 has several advantages with respect to currently used cooling techniques, such as avoiding the risk of infections and thrombosis associated with the use of intravascular devices where a solution is inserted into the blood stream resulting in change in a patient's fluid balance. Moreover, the inside of the patient's body can be heated and/or cooled at a rapid rate due to the large surface area within the colon.

Significant challenges exist, however, in placement of such devices into a patient's colon. The human colon (and the colon of other animals) includes various turns. FIG. 1 shows schematically the four regions of the human colon: the sigmoid colon, the ascending colon, the transverse colon and the descending colon. These regions are separated by at least four turns (labeled 1-4 in FIG. 1). As a result, a heat exchanger device needs to be able to be moved inside along the colon such as to advance over the turns in the colon. While the tube of the device disclosed by Balding's invention has at least one flex zone to promote bending of the device to allow for conformance of the device to the intestinal anatomy of the patient, it is believed that the highly flexible construction of the Balding device would render it quite difficult to move and ultimately properly place the heat exchange tube at a desired position within the patient's colon.

Body cooling devices and methods have particular application to those situations in which a reduction of blood supply to human body organs may lead to fatal ischemic injury, as may occur in many military and clinical settings including stroke, heart attack, traumatic hemorrhage, cardiac arrest, organ transplantation, and aortic aneurysm rupture. For example, traumatic hemorrhagic shock alone is responsible for over 35% of pre-hospital deaths. While extremity wounds are more amenable to compression to stop bleeding, 15% of Operation Iraqi Freedom and Operation Enduring Freedom battle injuries were to the torso (chest, abdomen, pelvis and back), where compression cannot be applied. Non-compressible torso hemorrhage (NCTH) is the leading cause of potentially survivable deaths of American troops. The control of bleeding is the only way to avoid the problems associated with massive hemorrhage in trauma. Resuscitative endovascular balloon occlusion of the aorta (REBOA) is a temporary maneuver for stopping NCTH. This technique involves inserting a balloon catheter to the appropriate section of the aorta, and inflating the balloon to occlude blood flow to the lower body, thus stopping the hemorrhage.

As a side effect, a “prolonged” usage of REBOA leads to fatal abdominal organ ischemic injury. Similarly, fatal abdominal ischemic injury occurs in many other clinical settings including cardiac arrest, organ transplantation, and aorta aneurysm rupture. Currently, there is no active intervention for the prevention of fatal abdominal organ ischemic damages. Recent studies show that in abdominal organs, such as the intestines and spleen, inflammatory response can lead to local and distant tissue or organ damage, known as systemic inflammatory response syndrome (SIRS). SIRS can occur either due to abdominal organ damage, damage to distant organs such as the brain, sepsis, severe sepsis, septic shock, and multiple organ dysfunction syndrome (MODS). SIRS can also lead to acute respiratory distress syndrome (ARDS) or acute lung injury (ALI).

Hypothermia (HT) is the most effective strategy currently known for ischemic organ protection. Its mechanism of action involves upregulating cell survival while inhibiting inflammatory and cell death activities. Use of deep cooling at approximately between zero and four degrees Celsius (° C.) together with organ-preservation solutions can protect the transplantation organ from ex vivo “ischemic injury” for up to 24-36 hours. Whole body deep cooling to between 10° C. and 15° C. in large animal models can offer optimal protection from lethal hemorrhagic shock (HS). In aggregate, the current literature suggests that optimized hypothermia is the best strategy to preserve organs from otherwise “irreversible” ischemic injury. In 2007, the FDA approved the first device specifically designed to ameliorate perinatal hypoxic ischemic brain damage. However, there are several challenges for implementing therapeutic hypothermia for adult organ ischemic patients: (i) cooling must be initiated as rapidly as possible for obtaining maximum protection, which is difficult to achieve in humans because even with an invasive endovascular device, cooling a human body even to 33° C. can take more than 1 hour; and (ii) whole body HT is often associated with myocardial dysfunction, pneumonia, and shivering, so that its role in traumatic hemorrhagic shock patients remains controversial.

Local and systemic inflammation occurs in many life threatening medical conditions. For example, acute pancreatitis is manifested by significant tissue swelling, local and systemic inflammation, and, in a severe case, pancreatic tissue necrosis. Diagnosis is mainly based on a CT scan. The acute form of the disease may become worse within 3 to 8 hours. No specific treatment for acute pancreatitis is currently available, so doctors can only provide supportive managements. The poorest results (i.e., a mortality rate of 64%) are found in patients with total necrosis of the pancreas, while partial necrosis of the pancreas had a relatively better outcome (i.e., a 33% mortality rate). Acute pancreatitis caused approximately 275,000 hospitalizations in 2009 (twice the number of 1988) and is the single most frequent gastrointestinal cause of hospital admissions in the U.S. About 20% of acute pancreatitis patients develop pancreatic tissue necrosis.

The annual global incidence of acute pancreatitis ranges between 13 and 45 per 100,000 persons according to 10 population-based cohort studies. The global estimates of incidence of acute pancreatitis are between 33 and 74 cases per 100,000 person-years, which causes between 1 and 60 deaths per 100,000 person-years. Variations in the above estimates result from differences in study methodology, difficulties in establishing accurate diagnoses, the use of different diagnostic criteria, and local lifestyle risk factors.

The above information disclosed in this background section is only for enhancement of understanding of the background of the invention and therefore it may contain information that does not form any part of the prior art.

### SUMMARY OF THE INVENTION

Disclosed herein are devices and methods configured to address one or more of the above described disadvantages of the prior art. However, achieving the above purposes and/or benefits is not a necessary feature to each of the exemplary embodiments, and the claims herein may recite subject matter that does not achieve the above stated purpose.

Body temperature management devices and methods are disclosed including a heat transfer or heat exchange device that is positionable within a patient's body to execute and affect heat transfer within the patient's body. For example, such devices and methods may be used to provide Focal Abdominal Cavity Cooling (FACC) to mitigate organ ischemic-reperfusion injury. Studies undertaken by the inventors herein in a rat model of hemorrhagic shock indicate that devices configured in accordance with at least certain aspects of the embodiments set forth herein dramatically reduce mortality as well as lethal organ damage after hemorrhagic shock, and thus provide enough time for local treatment of the patients or for transporting patients to trauma facilities. The devices and methods described herein offer unique advantages, including one or more of the following: (i) the devices and methods set forth herein are minimally invasive and can be deployed by properly trained personnel to induce profound FACC rapidly; (ii) in combination use with REBOA, the devices and methods set forth herein can keep adequate warm circulation to the brain, lungs, and heart; and (iii) FACC preserves abdominal organs from ischemia-reperfusion injury.

In accordance with certain aspects of an embodiment of the invention, a heat transfer system is provided that includes a heat exchange device. The heat exchange device may include a flexible conduit having at least two interior heat distribution channels, which channels carry a heat transfer medium (e.g., gas or fluid) therein. The heat exchange device may also include a steerable and preferably stiffness-adjustable guidewire, and preferably a bending adaptable segment configured to prevent the steerable guidewire from bending upon itself. The heat exchange device may further include one or more sensors, a locomotion system, an expandable outer membrane, and expandable outer membrane enforcement structure, at least two elongated and preferably cylindrical lumens or channels, and at least one irrigation lumen.

In accordance with still further aspects of an embodiment of the invention, a method is provided for the internal temperature management of a patient, such as via either colo-rectal or stomach-duodenum. The method may include providing a heat exchange device configured generally as discussed above, inserting the heat exchange device into either the patient's rectum-colon or stomach-duodenum, and causing the heat exchange device to affect heat transfer between the heat exchange device and the patient's tissue.

In accordance with still further aspects of an embodiment of the invention, the heat exchange device may include a temperature management catheter that may be provided in one of a variety of shapes (e.g., cylindrical, ovular, ribbed or contoured, etc.) for intraperitoneal use, and more particularly for direct cooling or warming of internal organs, such as a patient's pancreas in order to treat varied medical conditions, such as pancreatitis.

In accordance with still further aspects of an embodiment of the invention, an embedded temperature management device may include a hard or soft surface pad or balloon catheter provided in of a variety of possible shape and made of temperature changeable materials for directly cooling or warming of internal organs, such as a patient's pancreas, intestines, liver, or kidneys for the treatment of ischemia-reperfusion injury, and for treatment of inflammation, such as pancreatitis. An embedded temperature management device may further include connecting tubes or electricity-conductive wires in communication with one or more regulators, temperature management sources, and control units configured to perform heat exchange, such as a circulating a heat exchange carrier (e.g., liquid nitrogen, liquid argon, liquid oxygen, or the like), a refrigeration system, and a Peltier device.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the principles of the invention.

FIG. 1 shows a schematic view of a colon of a human subject.

FIG. 2 shows a cross-sectional view of a heat transfer catheter system.

FIG. 3 shows a close-up, cross-sectional view of a portion of the heat transfer catheter system of FIG. 2.

FIG. 4 shows a cross-sectional view of a heat transfer catheter for use in the system of FIG. 2.

FIG. 4a shows a schematic view of the heat transfer catheter of FIG. 4 in communication with a fluid control unit and a camera.

FIG. 5 shows a cross-sectional view of another exemplary heat transfer catheter.

FIG. 6 shows a cross-sectional view of a heat transfer catheter for use in the system of FIG. 2.

FIG. 7 shows a side cross-sectional view of a heat exchange device in accordance with certain aspects of an embodiment of the invention.

FIG. 8(a) shows an end cross-sectional view of the heat exchange device of FIG. 7 in a compact, uninflated state.

FIG. 8(b) shows an end cross-sectional view of the heat exchange device of FIG. 7 in an inflated state.

FIG. 9 is a close-up, side, cross-sectional view of the distal end of the heat exchange device of FIG. 7.

FIG. 10 is a schematic view of an embedded temperature management device in accordance with further aspects of an embodiment of the invention.

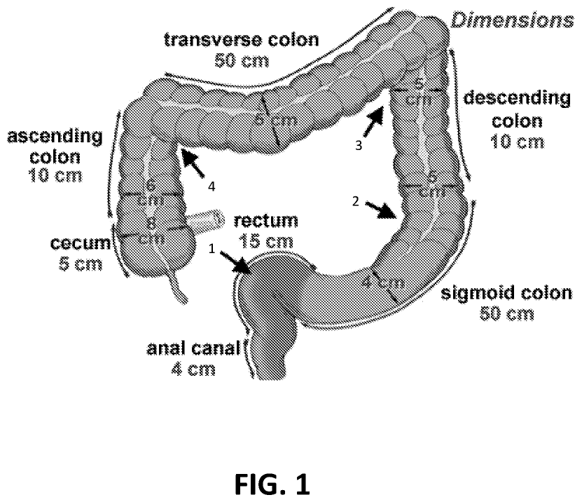

### DETAILED DESCRIPTION OF THE PREFERRED EMBODIMENTS

The following detailed description is provided to gain a comprehensive understanding of the methods, apparatuses and/or systems described herein. Various changes, modifications, and equivalents of the systems, apparatuses and/or methods described herein will suggest themselves to those of ordinary skill in the art. Descriptions of well-known functions and structures are omitted to enhance clarity and conciseness.

Hereinafter, an apparatus and method for performing temperature management of a subject's body is disclosed, and more particularly (with regard to a particular exemplary embodiment thereof) either colo-rectal or stomach-duodenum cooling or warming. Embodiments of the invention may, however, be configured in many different forms for various other body temperature management uses and should not be construed as limited to the exemplary embodiments set forth herein. Rather, these exemplary embodiments are provided so that this disclosure is thorough, and will fully convey the scope of the invention to those skilled in the art.

Throughout the drawings and the detailed description, unless otherwise described, the same drawing reference numerals are understood to refer to the same elements, features, and structures. The relative size and depiction of these elements may be exaggerated for clarity.

It will be understood that for the purposes of this disclosure, “at least one of X, Y, and Z” can be construed as X only, Y only, Z only, or any combination of two or more items X, Y, and Z (e.g., XYZ, XZ, XYY, YZ, ZZ). Further, it will be understood that when an element is referred to as being “connected to” another element, it can be directly connected to the other element, or intervening elements may be present.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item.

The use of the terms “first”, “second”, and the like does not imply any particular order, but they are included to identify individual elements. Moreover, the use of the terms first, second, etc. does not denote any order of importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Although some features may be described with respect to individual exemplary embodiments, aspects need not be limited thereto such that features from one or more exemplary embodiments may be combinable with other features from one or more exemplary embodiments.

This specification discloses devices and methods for controlling the temperature of a patient or an animal via, in accordance with certain aspects of an embodiment of the invention, a body temperature management device, such as a colon-rectal, a stomach-duodenum, or a peritoneally imbedded temperature management device. The devices and methods disclosed herein can be used for the therapy of hemorrhagic and septic shock, trauma, ischemia-reperfusion injury, or inflammatory disorders.

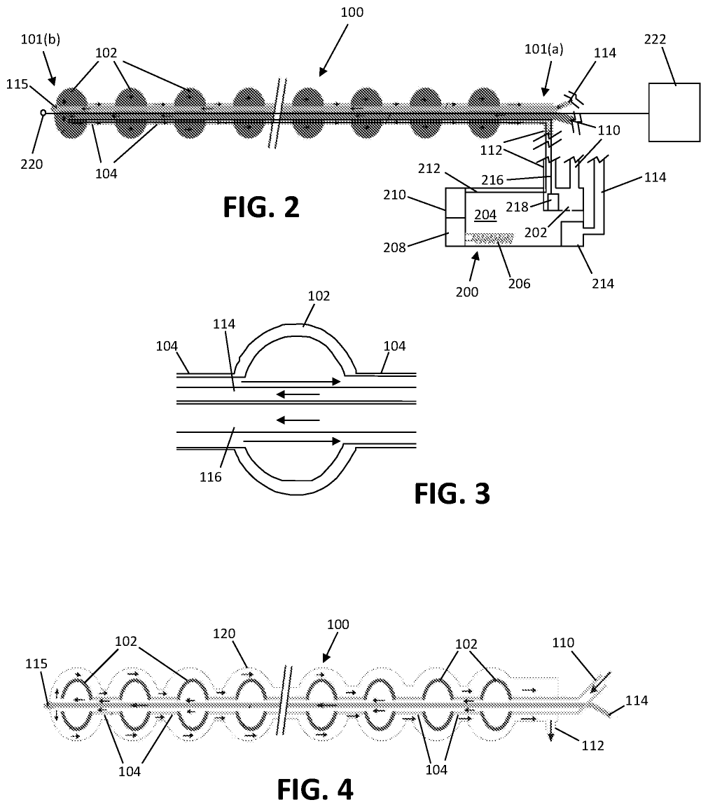

FIG. 2 shows a heat transfer catheter system including a heat transfer catheter (shown generally at 100) in fluid communication with a heat transfer fluid control unit (shown generally at 200). In accordance with a particular embodiment, heat transfer catheter 100 is formed as a colo-rectal temperature management catheter for use in a subject's body, and defines a heat transfer fluid carrier that transfers heat between a fluid within heat transfer catheter 100 and such portion of a patient's body in which heat transfer catheter 100 is placed, with such heat transfer being carried out through an outer surface of heat transfer catheter 100. Heat transfer catheter 100 includes a plurality of preferably oval-shaped, hollow nodes 102, and sections of flexible conduit 104 positioned between adjacent pairs of hollow nodes 102, which flexible conduit 104 allows fluid flow between and among nodes 102. In accordance with certain aspects of a particular embodiment, heat transfer catheter 100 may include a heat transfer fluid inlet line 110 that receives heat transfer fluid from fluid control unit 200, and a heat transfer fluid outlet line 112 that returns heat transfer fluid to fluid control unit 200 in a closed fluid circuit. In this configuration, heat transfer fluid flows into heat transfer catheter 100 from a proximal end 101(a), and flows toward distal end 101(b) of heat transfer catheter 100. At the distal end of heat transfer fluid inlet line 110, heat transfer fluid flows into and fills the interior of heat transfer catheter 100, allowing heat transfer to take place between the outer surface of heat transfer catheter 100 and a patient's body in which heat transfer catheter 100 is placed. The heat transfer fluid then continues to flow from the distal end of heat transfer catheter 100 to the proximal end thereof, until it exits at the proximal end through heat transfer fluid outlet line 112 for return to fluid control unit 200.

Preferably, an irrigation line 114 is also provided and extends from the proximal end of heat transfer catheter 100, through its entire length and to the distal end of heat transfer catheter 100, providing a fluidly isolated channel that may carry irrigation fluid, medication, or other materials through heat transfer catheter 100 for delivery to the patient's body, such as irrigation fluid to clean the patient's colon so as to improve heat transfer between the colon and heat transfer catheter 100.

Heat transfer fluid inlet line 110, heat transfer fluid outlet line 112, and irrigation line 114 may be formed of flexible or elastic materials typically used in catheters and known to those skilled in the art, such as latex, silicone, TEFLON, or the like. Likewise, heat transfer fluid inlet line 110, heat transfer fluid outlet line 112, and irrigation line 114 all connect to their respective connections on fluid control unit 200. With continuing reference to FIG. 2, fluid control unit 200 includes a pump 202 that pumps heat transfer fluid from a chamber 204, into heat transfer fluid inlet line 110 which carries the heat transfer fluid through the length of heat transfer catheter 100 from its proximal end 101(a) to its distal end 101(b), and then back through the interior of heat transfer catheter 100 until it exits through heat transfer fluid outlet line 112, and back into pump 202, all in a closed fluid circuit. Chamber 204 may include a heat exchange element 206 and a temperature regulator 208 of standard configuration and capable of controlling the temperature of heat transfer fluid that is delivered to heat transfer catheter 100. The temperature regulator 208 may, by way of non-limiting example, comprise a container with cold or hot fluid, a compressor device, an exothermic or endothermic device, a Peltier cooling device, a heating device, or a combination of the above.

Preferably, a processor 210 is also provided for maintaining temperature set-points for the heat transfer fluid and for controlling temperature regulator 208. A temperature probe 212 may extend through heat transfer catheter 100 and may communicate with processor 210 to ensure that the temperature of heat transfer fluid within heat transfer catheter 100 is maintained at an intended temperature, and so as to allow processor 210 to control temperature regulator 208 to maintain such temperatures within the established temperature set points. Such temperature probe 212 and regulator 208 are configured to closely control and maintain an accurate fluid temperature of heat transfer fluid within the patient's body in order to reduce the risk of cold or heat damage to the contacted tissue. Due to heat exchange between the heat exchange catheter 100 and the patient's body, the temperature of the circulating heat transfer fluid is significantly different between the inside and outside of the patient's body, and thus should be accurately controlled so that the fluid can perform accurate temperature exchange. For example, when using heat transfer catheter 100 as a rectal colon heat exchanger, the fluid in the intra-rectal colon segment of the device should be maintained at a temperature that is neither too hot nor too cold, thus avoiding the risk of heat or cold damage to the contacted colon tissue.

Further, a pressure sensor 216 may extend through heat transfer catheter 100 and may communicate with a pressure feedback controller 218 having stored thereon established pressure set points (that may be a part or function of processor 210 or separate therefrom) to ensure that the pressure within heat transfer catheter 100 does not exceed a predetermined threshold pressure that might cause rupture of the exterior of heat transfer catheter 100, which rupture could cause damage to the patient's tissue contacting heat transfer catheter 100.

Additionally, fluid control unit 200 may include irrigation unit 214 configured to store and pump irrigation fluid through irrigation line 114 as discussed above. While shown in FIG. 2 as physically attached to the remainder of fluid control unit 200, those skilled in the art will recognize that irrigation unit 214 may likewise be provided as a separate unit therefrom.

Those skilled in the art will recognize that the schematic view of fluid control unit 200 shown in FIG. 2 is exemplary only, and that any mechanisms for pumping and controlling the temperature of a heat exchange fluid to heat transfer catheter 100, and likewise for delivering irrigation fluid through heat transfer catheter 100, may be used without departing from the spirit and scope of the invention. For example, and with respect to each of the configurations described herein, fluid driving devices other than those described above, including by way of non-limiting example a syringe, may be used to deliver heat transfer fluid, irrigation fluid, or other fluids to and through heat transfer catheter 100 without departing from the spirit and scope of the invention.

As mentioned above, in accordance with certain aspects of an embodiment and with reference to the close-up cross-sectional view of FIG. 3, heat transfer catheter 100 may include oval shaped, hollow nodes 102, and sections of flexible fluid conduit 104 positioned between adjacent nodes 102 and allowing fluid communication between and among them. Importantly, nodes 102 are configured with greater rigidity and less flexibility than fluid conduit sections 104. More particularly, while sections of flexible conduit 104 between adjacent nodes 102 may compress during insertion into a patient's body, such as into the patient's colon, it is important that the heat transfer catheter 100 be able to maintain its overall elongate shape during such insertion to allow it to be pushed into the intended location in the patient's body without curling, pinching, or the like. It is also important, however, to ensure that heat transfer catheter 100 be able to bend as it traverses the patient's anatomy, such as their colon. Providing nodes 102 with greater rigidity than the other elements of heat transfer catheter 100 achieves, at least in part, these results. Specifically, nodes 102 are provided sufficient rigidity (and specifically greater rigidity than intermediate sections of flexible conduit 104) so as to not compress during insertion into the patient's body, such as their colon, and instead each node 102 will serve to push an adjacent node further into the patient's colon during insertion of heat transfer catheter 100, while intermediate sections of flexible conduit 104 allow heat transfer catheter 100 to bend to adapt to the patient's particular anatomy. The dimensions of oval nodes 102 are not critical, and can change depending upon the subject in which heat transfer catheter 100 is to be placed so as to best adapt to the specific anatomy of that subject, although the cross-section of each node 102 should have a major axis that is larger than a perpendicular minor axis.

Oval shaped, hollow nodes 102 and sections of flexible conduit 104 may be formed of any suitable, biologically compatible material, such as latex, silicone, TEFLON, or any other biologically inert materials that will allow heat transfer between the inside of heat transfer catheter 100 and the patient's body in which heat transfer catheter 100 is placed. In order to provide nodes with greater rigidity than interconnecting sections of flexible conduit 104, as shown in the close-up sectional view of a portion of heat transfer catheter 100 of FIG. 3, nodes 102 may (by way of non-limiting example) be formed of the same material as conduit 104, but may have a thickness significantly greater than flexible conduit 104, thus providing nodes 102 with greater rigidity than conduit 104. Alternatively, nodes 102 may be formed of a different biologically inert material having greater rigidity than conduit 104, or of the same material but with reinforcement that increases the rigidity of nodes 102 beyond that of flexible conduit 104. In a particularly preferred configuration, nodes 102 may be positioned approximately 1-10 millimeters apart from one another so as to optimally adapt to typical colon structures. However, the size, shape and number of nodes 102 and sections of flexible conduit 104 may be readily adjusted to best fit the colon or other anatomical structures of a given patient (human, animal, multicellular organism, etc.).

With this configuration of nodes 102 having greater rigidity than intermediate sections of flexible conduit 104, heat transfer catheter 100 may be more easily placed into difficult-to-navigate areas of a patient's body, such as by way of non-limiting example a patient's colon, and more particularly may more easily pass the turning areas in the sigmoid, ascending, transverse, and descending colon, all while maintaining the overall length of heat transfer catheter 100 so as to avoid it being propelled backward by pressure or bowel movements.

Moreover, while the above exemplary discussion focuses on use of the heat transfer catheter 100 for rectal colon heat transfer, heat transfer catheter 100 may likewise be used in other areas of the patient's body, such as (by way of non-limiting example) for upper body cavity cooling, in which the heat transfer catheter 100 is placed in the patient's esophagus, stomach, or upper digestive tract, with heat transfer being carried out at such location to better focus heating or cooling where specifically needed for a given patient condition.

The heat transfer fluid carried through heat transfer catheter 100 may comprise any material or combination of different materials, either fluid, oil, or viscous, including but not limited to water, a physiological fluid such as Ringer's solution, a chemical fluid, a solvent, a biological fluid, a therapeutic fluid, a lubricant, and combinations of the foregoing or similarly configured fluids. As explained above, heat transfer inlet line 110 and heat transfer outlet line 112 are connected to temperature-controlling apparatus of fluid control unit 200 to enable the circulation of the fluid from the temperature-controlling apparatus to heat transfer fluid inlet line 110, through the length of heat transfer catheter 100 from its proximal end 101(a) to its distal end 101(b), and further through the interior of heat transfer catheter 100 from its distal end 101(b) to its proximal end 101(a) (outside of heat transfer fluid inlet line 110), further to heat transfer fluid outline line 112 and then back to the temperature-controlling apparatus. The temperature exchange fluid may be driven either via pump 202 or any other fluid handling device, such as (by way of non-limiting example) a syringe, to circulate the fluid or mix of different fluids. Arrows on FIGS. 2 and 3 show the flow path of fluids through their respective flow paths in heat transfer catheter 100. The heat transfer fluid may be driven continuously.

Irrigation line 114 may extend the full length of heat transfer catheter 100, and may include an irrigation line outlet 115 that may have an oval shaped structure. As is the case with heat transfer fluid inlet line 110, irrigation line 114 may extend through the interior of nodes 102 and sections of flexible conduit 104, extending essentially parallel to heat transfer fluid inlet line 110. The function of irrigation line 114 is to perform lubrication, enema, or to remove the contents from the patient's colon rectum lumen. Irrigation fluid may be injected or withdrawn via the inlet through the span of irrigation line 114 and further to the outlet 115, thereby irrigating the materials in the patient's colon. As explained above, the irrigation fluid may be driven either via a pump in irrigation unit 214, or via a device such as a syringe to perform the irrigation or lubrication function. The irrigation fluid may be made of any materials or any combination of different materials, either fluid, oil, or viscous, including but not limited to water, a physiological fluid such as Ringer's solution, a chemical fluid, a solvent, a biological fluid, a therapeutic fluid, lubricant, gases, or a combination of the above.

Optionally, and with continued reference to FIG. 2, an endoscopic camera 220 may also be provided attaching to and optionally extending through and out of the distal end 101(b) of heat transfer catheter 100, the proximal end of camera 220 connecting to a video screen 222 to aid the clinician in properly placing heat transfer catheter 100 at the desired location within the patient by visualizing the body cavity (e.g., the patient's intestine) in which the heat transfer catheter 100 is being placed.

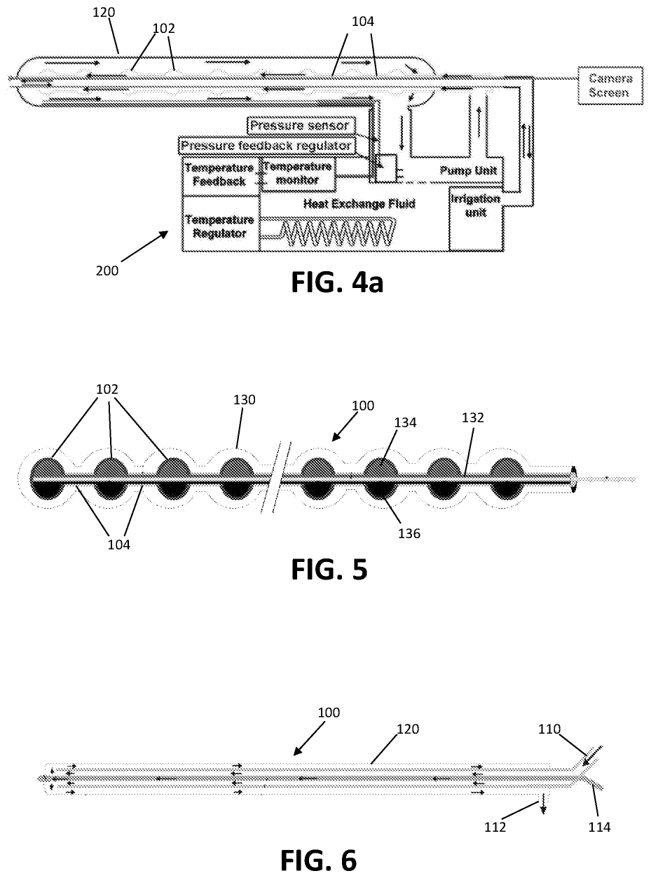

Referring next to FIG. 4 (fluid control unit 200 omitted for clarity), heat transfer catheter 100 may also include an inflatable, flexible or elastic balloon-shaped membranous tube 120. If such a balloon 120 is provided, heat transfer fluid inlet line 110 leads directly and attaches to the proximal-most section of flexible conduit 104, thus carrying the heat transfer fluid delivered to the proximal end of heat transfer catheter 100 toward the distal end of heat transfer catheter 100 through the interiors of nodes 102 and sections of flexible conduit 104. At the distal end of heat transfer catheter 100, the heat transfer fluid exits the distal-most node 102, and thereafter flows from the distal end of heat transfer catheter 100 to the proximal end of heat transfer catheter 100 between the exterior of nodes 102 and sections of flexible conduit 104, and the interior of balloon 120. At the proximal end of heat transfer catheter 100, the heat transfer fluid exits from balloon 120 through heat transfer fluid outlet line 112. The directional arrows on FIG. 4 show the direction of fluid flows through heat transfer catheter 100. FIG. 4a provides a schematic view of the heat transfer catheter 100 shown in FIG. 4 in combination with fluid control unit 200 configured as described above.

Balloon 120 should be sufficiently thin so as to ensure efficient heat transfer between heat transfer catheter 100 and the patient's tissue, but it is likewise important to ensure that balloon 120 is of sufficient strength so as to protect against breakage or rupture and thus potential damage to the patient's tissue. Thus, balloon 120 may be formed of more than one layer of thin membrane material to help protect against potential leakage while maintaining efficient heat transfer.

Once again, irrigation line 114 is provided, extending from the proximal end of heat transfer catheter 100 to and through the distal end thereof so as to provide irrigation fluid from its tip 115 to the patient's colon (or other such anatomy in which heat transfer catheter 100 is placed).

Next, according to further aspects of an embodiment and with reference to FIG. 5, heat transfer catheter 100 may be equipped to contain an endothermic or exothermic reaction to effect heat transfer, instead of receiving a heat transfer fluid from fluid control unit 200. As used herein, the term “endothermic process” refers to a process or reaction in which the system absorbs energy (e.g., absorption of heat from the surroundings), and the term “exothermic process” refers to a process or reaction in which the system releases energy (e.g., in the form of heat). In order to contain such processes, nodes 102 and sections of flexible conduit 104 configured as described above form a sealed compartment containing reactants (as discussed further below), and are optionally contained within a closed external membrane 130 formed of flexible material, such as the balloon 120 of FIG. 4, with such closed external membrane 130 serving to prevent leakage of materials from nodes 102 and flexible conduit sections 104. In this configuration, the heat transfer fluid carrier is thus defined by nodes 102 and flexible conduit sections 104. Such heat transfer fluid carrier is separated into two or more compartments by one or more separating membranes 132. Those separate compartments contain different materials, such as a first reactant 134 and a second reactant 136 that, when combined or in contact with one another, carry out an endothermic or exothermic reaction.

Reactants 134 and 136 may comprise different endothermic or exothermic materials or any combination of different materials in either powder, particle, solid, fluid, oil, or viscous form. An example of a suitable configuration for use with the devices and methods set forth herein to allow an endothermic reaction include selecting [Ba(OH)₂ 8H₂0] as reactant 134, and [(NH₄)(NO₃)] as reactant 136, although those skilled in the art will recognize that various endothermic reactions (and corresponding materials) are currently available and may be used without departing from the spirit and scope of the invention. Likewise, an example of a suitable configuration for use with the devices and methods set forth herein to allow an exothermic reaction include selecting a small amount of notched ferrous metal as reactant 134 and a supersaturated solution of sodium acetate (3H₂ CH₃COONa) in water as reactant 136, although those skilled in the art will recognize that various exothermic reactants (and corresponding materials) are currently available and may be used without departing from the spirit and scope of the invention.

The separating membrane 132 is removable or breakable at the time that the endothermic or exothermic reaction is desired to be carried out, such that reactants 134 and 136 will mix with one another within the heat transfer fluid carrier. Separating membrane 132 may be made of either strong or fragile materials which may be readily selected by those of ordinary skill in the art. If the separating membrane 132 is made of strong materials, then the separating membrane 132 may be pulled to cause the mixing of reactants 134 and 136. If the separating membrane 132 is made of fragile materials, then the separating membrane 132 may be broken by squeezing the heat transfer fluid carrier, thereby causing the mixing of reactants 134 and 136. The orientation of separating membrane 132 may be at any plane in the interior of the heat transfer fluid carrier. Optionally, separating membrane 132 may also comprise a small bag or other container holding a small amount of one of reactants 134 or 136, with the remaining reactant held within the rest of the internal space of the heat transfer fluid carrier.

As used with regard to the embodiment shown in FIG. 5, the term “heat transfer fluid” is intended to include the reactants 134 and 136 shown in FIG. 5.

Next, with reference to FIG. 6 and in accordance with certain aspects of an embodiment (fluid control unit 200 being omitted for clarity), a cylindrical heat transfer catheter 100 may be provided of sufficient rigidity so as to allow its placement within the colon of a patient while enabling its navigation through the turns of the patient's sigmoid, ascending, transverse, and descending colon. In this configuration, heat transfer fluid inlet line 110 extends from the proximal end of heat transfer catheter 100 to the distal end thereof, and while round, otherwise has a wall configuration identical to that described above for nodes 102 so as to provide fluid inlet line 110 with sufficient rigidity to allow its navigation through the patient's body to its intended location. The distal end of heat transfer fluid inlet line 110 opens to the interior of cylindrical balloon 120, directing the heat transfer fluid from heat transfer fluid inlet line 110 to the interior of cylindrical balloon 120, and back to the proximal end of heat transfer catheter 100, where the heat transfer fluid again exists through heat transfer fluid outline line 112. Irrigation line 114 may again be provided, extending through heat transfer fluid inlet line 110 and exiting cylindrical balloon 120 in the same manner as described above. The directional arrows on FIG. 6 show the fluid flows through heat transfer catheter 100.

With continued reference to the embodiment reflected in FIG. 6, cylindrical balloon 120 is preferably formed with a rounded, smooth distal tip so as to aid in its insertion into the patient's body.

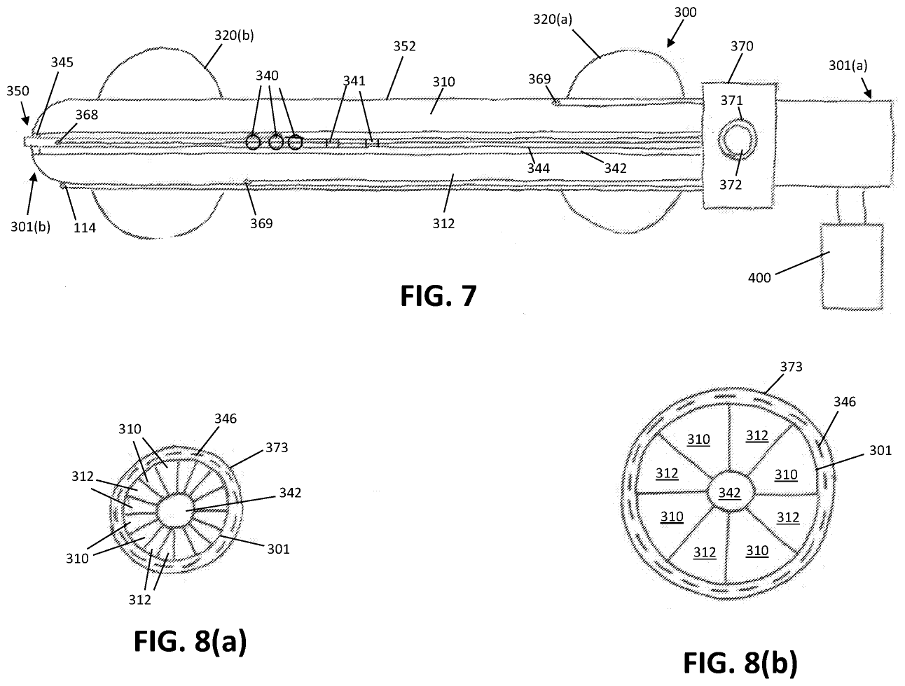

Next, FIG. 7 shows a side, cross-sectional view of a heat transfer device in accordance with certain aspects of an embodiment of the invention. In this configuration, a heat transfer system is shown including a heat exchange device 300 in fluid communication with a heat transfer medium carrier control unit 400 (which may be configured to function in the same manner as heat transfer fluid control unit 200 discussed above). In this configuration, heat exchange device 300 may form a colo-rectal, a stomach-duodenum, or a peritoneally embedded temperature management device for use in a subject's body. Heat exchange device 300 defines a heat transfer medium (e.g., fluid or gas) carrier that transfers heat between the heat transfer medium within heat exchange device 300 and such portion of a patient's body in which heat exchange device 300 is placed, with such heat transfer being carried out through an expandable outer membrane 352 of heat exchange device 300.

With reference to FIGS. 7, 8(a) and 8(b), heat exchange device 300 preferably includes flexible channels, and more particularly at least one inlet channel 310 and one outlet channel 312, positioned radially between a guidewire lumen 342 and the expandable outer membrane 352 of heat exchange device 300. A guidewire 144 extends through guidewire lumen 342 for aiding in placement of the heat exchange device 300 in the patient's body. Expandable outer membrane 352 allows heat transfer between the heat transfer medium within the heat exchange device 300 and the patient's tissue that is to be treated by heat exchange device 300. A steerable control apparatus 370 is provided, and mechanically connects to a steerable tip (shown generally at 350) of the heat exchange device 300 to aid in navigating the heat exchange device 300 through the patient's body. Steerable tip 350 and the expandable outer membrane 352 of heat exchange device 300 are configured as to allow an operator to insert heat exchange device 300 within the turns of portions of a patient's internal organs, such as those located at the sigmoid colon, between the ascending colon and the transverse colon, and between the transverse colon and the descending colon, with the expandable outer membrane 352 expanding to a diameter that contacts the walls of the colon to provide cooling. An irrigation lumen 114 configured as described above may be provided, and may for example be positioned on an external surface of the heat exchange device 300, or may alternatively be provided as a separate lumen extending through the interior of heat exchange device 300. Further and as described above, heat exchange device 300 may include an endoscopic camera, a light, and sensors all configured as discussed in the embodiments set forth above. The foregoing features will now be discussed in greater detail below.

In the exemplary configuration of FIGS. 7, 8(a) and 8(b), heat transfer fluid flows into heat transfer device 300 from a proximal end 301(a), and flows toward distal end 301(b) of heat transfer device 300 through one or more inlet channels 310. At the distal end 301(b) of heat transfer device 300, heat transfer medium flows from each inlet channel 310 into a fluidly connected outlet channel 312, and returns to control unit 400 through outlet channels 312. Preferably, each inlet channel 310 fluidly connects with an adjacent outlet channel 312 at distal end 301(b) of heat exchange device 300. As such flow proceeds, heat transfer fluid fills the interior of both inlet and outlet channels 310 and 312, respectively, causing expandable outer membrane 352 of heat exchange device 300 to expand as the heat exchange device 300 inflates from the configuration shown in FIG. 8(a) (in which each inlet channel 310 and outlet channel 312 is folded into a collapsed wall configuration) to the inflated configuration shown in FIG. 8(b). In such inflated configuration, heat exchange device 300 comes into contact with the patient's tissue so as to allow heat transfer to take place between expandable outer membrane 352 of heat transfer device 300 and the patient's tissue to be treated.

While one each of inlet channel 310 and outlet channel 312 may be sufficient to render heat exchange device 300 operable, in a particularly preferred configuration, a plurality of inlet channels 310 and outlet channels 312 are provided as shown in FIGS. 8(a) and 8(b). Such configuration increases the uniformity of the heat exchange along the heat exchange device 300 and increases the symmetry of the expandable outer membrane 352 as it expands. Inlet channels 310 and outlet channels 312 are preferably oriented radially and generally symmetrically around guidewire lumen 342. Preferably, the inlet channels 310 and outlet channels 312 also alternate circumferentially around guidewire lumen 342.

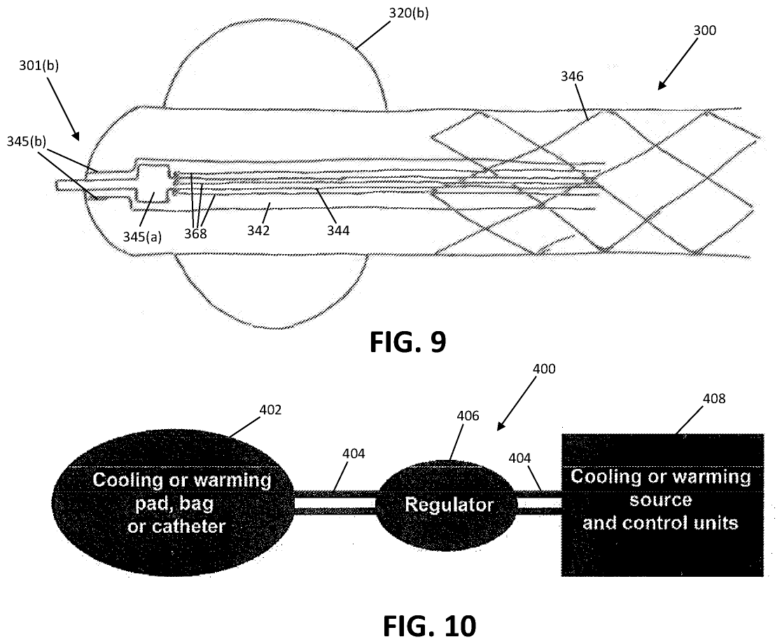

With continued reference to FIGS. 7, 8(a) and 8(b), guidewire lumen 342 receives a guidewire 344 and preferably at least one sensor. Guidewire 344 engages (such as at 345) heat exchange device 300 at a distal end 350 to transfer force from guidewire 344 to heat exchange device 300. More particularly and with reference to the close-up sectional view of FIG. 9, guidewire 344 may include an enlarged head 345(a) positioned a distance away from the distal tip of guidewire 344, which enlarged head 345(a) engages a narrowed end 345(b) of guidewire lumen 342. Alternatively, distal tip of guidewire 344 may removably engage the distal end 301(b) of heat exchange device 300, such as through a threaded engagement. In each case, guidewire lumen 342 is preferably a generally cynlindrical lumen that is configured to transmit forces from the guidewire 344 (as controlled by steerable control apparatus 370) by a push-pull technique. More particularly, the steerable control apparatus 370 includes a set of control wires 368 fixedly attached to guidewire 344 at various positions along the circumference and optionally the length of guidewire 344, each of which control wires 368 connect to steerable control apparatus 370. Through manipulation of steerable control apparatus 370, distal end 301(b) of heat exchange device 300 may bend in a desired direction within the patient's colon, thus rotating the tip 350 and preventing undesired tension of guidewire 344. In an exemplary embodiment, steerable control apparatus 370 may include two or more dials that each rotate in separate directions to control the direction of the tip 350 of heat exchange device 300. For example, a first dial 371 rotates to push and pull on some of the wires 368 to steer the tip 350 in a first direction, and a second dial 372 rotates to push and pull on other wires 368 to steer the tip 350 in a second direction that is generally orthogonal to the first direction. Rotating the dials in combination allows the physician to steer the tip 350 in many directions. Thus, the steerable control apparatus 170 is configured to steer the tip 350 in many directions. However, additional dials or the like may be provided to further precisely steer portions of the heat exchange device 300, including the tip 350, in many other directions without departing from the spirit and scope of the invention.

Guidewire 344 thus steers heat exchange device 300 from within the body of heat exchange device 300 (and more particularly from within guidewire lumen 342). Guidewire lumen 342 is a generally rigid or semi-rigid lumen that is positioned centrally of heat transfer inlet channels 310 and outlet channels 312, such as along the central axis of heat exchange device 300. Guidewire 344 may further include a camera and light (configured as discussed in the embodiments set forth above), and sensors including one or more of a pressure sensor, a temperature probe, and at least one medium flow sensor (discussed in greater detail below).

Guidewire 344 may include stiffness structures 340 and/or adjustable stiffness mechanisms 341 that are configured to ease insertion of the heat exchange device 300 into the deep turns of the patient's colon or duodenum. Stiffness structures 340 are particularly configured to prevent heat exchange device 300 from bending to an acute angle, such that the heat exchange device 300 would bend backwards upon itself. Stiffness structures 340 and/or adjustable stiffness mechanisms 341 are positioned along guidewire 344 between the steerable tip 350 and proximal end 301(a) of the heat exchange device 300. In this configuration, when the heat exchange device 300 contacts a wall of the colon, the reaction force from the wall of the colon forces the distal end 301(b) forward within the colon, as desired to insert the heat exchange device 300. However, the stiffness structures 340 are configured to provide a portion of guidewire 344 the ability to passively flex when in contact with objects, such as angular pressures of the colonic wall of the patient. For example, the portion of guidewire 344 having stiffness structures 340 may bend passively by receiving an external force to gradually increase the curvature of the distal end 301(b) of device 300 relative to the proximal end 301(a).

In a particularly preferred embodiment, stiffness structures 340 may comprise rigid masses affixed to the guidewire to limit bending or compression in the region of such rigid masses. Likewise, while a variety of mechanisms are available and known to those skilled in the art for providing a guidewire with adjustable stiffness, adjustable stiffness mechanisms 341 may, in a particularly preferred embodiment, comprise spring structures within the body of the guidewire that may be tightened. Moreover, guidewire 344 may optionally be removable from heat exchange device 300, and may be formed of a generally medically-approved material, such as a PEEK plastic or metal, or the like, thus allowing its removal and sterilization for reuse.

Irrigation lumen 114 extends from proximal end 301(a) of heat exchange device 300 to the distal end 301(b) of heat exchange device 300 for providing irrigation fluid for lubrication, an enema, or to remove the contents of the patient's colon. As shown in FIG. 7, irrigation lumen 114 may be positioned at the outer diameter of the expandable outer membrane 352 of heat exchange device 300, or alternatively may be positioned on the interior of heat exchange device 300 as a separate and independent lumen from inlet channels 310 and outlet channels 312. Irrigation lumen 114 may alternatively carry medication or other materials through the entire length of heat exchange device 300 for delivery to the patient's body, such as intestinal lumen protective substances to protect the patient's colon or duodenum so as to improve heat transfer between the colon and heat exchange device 300, and preserve tissue integrity.

Inlet channels 310, outlet channels 312, and irrigation lumen 114 may be formed of flexible or elastic materials typically used in catheters and known to those skilled in the art, such as latex, silicone, TEFLON®, or the like. Likewise, inlet channels 310, outlet channels 312, and irrigation lumen 114 all connect to their respective connections on control unit 400. As with the embodiments discussed above, control unit 400 may include a pump, such as a circulating pump, a syringe, or the like, that pumps heat transfer medium from a chamber into heat transfer medium inlet channels 310 that carry the heat transfer medium through the heat exchange device 300 from its proximal end 301(a) to its distal end 301(b). The heat transfer medium returns to the proximal end 301(a) through the outlet channels 312 and exits at proximal end 301(a) into control unit 400, all in a closed fluid circuit.

Expandable outer membrane 352 of heat exchange device 300 is configured to expand the diameter of the device 300 to directly contact the device 300 with the patient's tissue, such as by way of non-limiting example the patient's colon wall, to conductively exchange heat between such tissue and the device 300, although other forms of heat exchange are feasible, such as radiant heat exchange and the like. The expandable outer membrane 352 is preferably formed of an elastic material, such as rubber, latex, TEFLON®, silicone, medical-grade plastic, or the like, to expand to the desired diameter and contain the heat exchange fluid and irrigation fluid, as described above. Furthermore, the expandable outer membrane 352 may be disposable or configured to be reusable, such that it can be separated from the electronic sensors and sterilized.

Expandable outer membrane 352 is preferably a thin membrane to facilitate heat exchange. However, and with reference to the close-up view of heat exchange device 300 of FIG. 9, a reinforcement structure 346 may also be provided that is configured to prevent the thin expandable outer membrane 352 from breaking or tearing within a patient's colon or duodenum. The reinforcement structure 346 is generally similar to a stent, a spring wire mesh (such as a braided or woven spring mesh), an elastic rubber, or the like, that encircles at least a portion of the expandable outer membrane 352. The reinforcement structure 346 may also be configured to prevent the expandable outer membrane 352 from expanding to a larger than desired diameter, which could result in placing too much pressure on the patient's colon or duodenum.

Further, and with reference to FIGS. 8(a) and 8(b), expandable outer membrane 352 may optionally be covered by a removable sheath 373 to protect the expandable outer membrane 352 when it is outside of a patient and when it is being inserted into a patient. Once the heat exchange device 300 is placed into a patient's colon, the sheath 373 can be pulled or slid from the expandable outer membrane 352 to allow the expandable outer membrane 152 to expand as described above. Removable sheath 373 may be formed of a light, thin plastic or elastic material, or the like.

In certain configurations, heat exchange device 300 may optionally be provided a proximal balloon 320(a) and a distal balloon 320(b), which balloons may be inflated to hold portions of device 300 against the patient's colon during the insertion process, and to optionally enlarge the colon during the insertion process. Proximal balloon 320(a) is positioned near the proximal end 301(a) of the heat exchange device 300, and distal balloon 320(b) is positioned near the distal end 301(b) of the heat exchange device 300. Balloons 320(a) and 320(b) are preferably formed of an inflatable, flexible, or elastic membrane tube, and are sufficiently thin so as to ensure efficient heat transfer between the epxandable outer membrane 352 of heat exchange device 300 and the patient's tissue. Nonetheless, to protect against risk of breakage or rupture, balloons 320(a) and 320(b) may be formed of more than one layer of thin membrane material to help protect against potential leakage while maintaining efficient heat transfer.

Balloons 320(a) and 320(b) may be inflated sequentially or simultaneously, as described in greater detail below, to move heat exchange device 300 forward or backward within the portions of the patient's colon or duodenum. Optionally, a movement aid such as a spring (not shown) attached to each of balloons 320(a) and 320(b) may be provided to assist in moving heat exchange device 300 within portions of the patient's colon or duodenum. Preferably, a separate lumen is provided to each of balloon 320(a) and 320(b) for independent inflation and deflation of the balloons, such as from an air or other gas or fluid source within control unit 400. Likewise, air or fluid ports 369 (configured in like fashion to the outlet of irrigation lumen 114 described above) are provided to inject or withdrawal air or fluid (e.g., the same irrigation fluid that is delivered to the outlet of irrigation lumen 114) into or from the space between the heat exchange device 300 and the inner surface of the rectum, colon, or duodenum to move the heat exchange device 300 forward or backward within portions of the intestine, as further described below. The balloons 320(a) and 320(b) when inflated contact the surface of the patient's tissue to be treated, such as the inner surface of the patient's intestine to provide a reaction force against the patient's intestinal walls, and in combination with a spring member (if provided) and air or fluid from ports 369, assist in moving the heat exchange device through the patient's colon.

More particularly, irrigation fluid provided through ports 369 may act as a propelling force to move heat exchange device 300 within the patient's intestine. To assist in propelling heat exchange device 300 further into the intestine of a patient, each of balloons 120(a) and 120(b) may be inflated to contact the inner surface of the intestine to provide a seal. Irrigation fluid, air, or other fluid fills the intestine lumen from ports 369. Distal balloon 120(b) is configured to translate or slide along small distances on heat exchange device 300, such as on and near the steerable tip 350. The proximal balloon 320(a) is preferably fixed on the heat transfer device 300. Thus, irrigation fluid, for example, fills the space between the inner surface of the patient's colon and the heat exchange device 300 between balloons 320(a) and 320(b), such that filling the colon with additional irrigation fluid increases the irrigation fluid pressure that is exerted on the balloons 320(a) and 320(b), the expandable outer membrane 352, and the inner surface of the patient's intestine. Because distal balloon 320(b) is movable, it will translate further into the patient's intestine, such as a few millimeters or centimeters, under the irrigation fluid pressure. When the distal balloon 320(b) is in the desired position, the fluid control unit 400 removes the irrigation fluid through ports 369 to decrease the irrigation pressure. Proximal balloon 320(a) is then deflated, and the heat exchange device 100 translates within the intestine as the now advanced distal balloon 320(b) pulls the heat exchange device 100 further into the patient's colon. Further and as discussed above, heat exchange device 300 is steerable using the push-pull steerable control apparatus 370. Thus, heat exchange device 300 may be inserted into the patient's colon by repeatedly pressurizing the irrigation fluid and expanding and deflating balloons 320(a) and 320(b).

As discussed above, control unit 400 controls the temperature of heat exchange device 300, and may comprise a container with cold or hot fluid or gas, a compressor device, an endothermic device, a Peltier cooling device, or a heating device. The sensors of heat exchange device 300 may include at least one pressure sensor, a temperature probe, and at least one flow sensor, each of which may be of standard configuration and are thus not further detailed here. Such sensors are positionable at various and multiple locations along heat exchange device 300, and are particularly positioned to preferably provide measurements at multiple points of the heat exchange inlet channels 310 and heat exchange outlet channels 312. For example, in one configuration, pressure sensors and flow sensors may be provided near proximal end 301(a) and distal end 301(b) of heat exchange device 300, and near those portions of heat exchange device 300 that will undergo bending and turning during use, in order to avoid over-pressuring the heat exchange device 300. Further, multiple temperature probes may be provided to closely measure the internal temperature of the intestine and to control the heat exchange.

Such temperature probes may extend through heat exchange device 300 and communicate with the processor in control unit 400 to ensure that the temperature of the heat transfer fluid within heat exchange device 300 is maintained at an intended temperature. The processor and temperature probes may control the temperature of the heat exchange fluid between a range of 0° C. and 60° C., as necessary for the patient and as determined by a physician. However, the processor and temperature probes may control the temperature of the heat exchange medium within narrower temperature ranges, or at different temperatures, such as negative Celsius temperatures, depending upon the chosen heat exchange medium.

Further and as discussed with respect to the embodiments above, the pressure sensor of heat exchange device 300 may communicate with the pressure feedback controller of control unit 400 to ensure that the pressure within heat transfer device 300 does not exceed a predetermined threshold pressure that might cause rupture of the exterior of heat exchange device 300, which rupture could cause damage to the patient's tissue contacting heat exchange device 300. However, pressure within heat exchange device 300 may be increased to expand the diameter of the expandable outer membrane 352 to contact the walls of the colon, as described above. By way of non-limiting example, heat exchange device 300 may be expandable to a diameter of approximately 20 centimeters.

Thus and in general, control unit 400 is configured to regulate at least the temperature and pressure of the heat exchange medium within heat exchange device 300. Control unit 400 receives input from the sensors discussed above, including by way of non-limiting example a pressure sensor, temperature probe, and at least one fluid flow sensor, to determine when the heat exchange medium within a patient's body is significantly different from the temperature of the heat exchange fluid outside of the patient's body. The control unit 400 adjusts the temperature of the heat exchange fluid, such as by cooling or warming the heat exchange fluid, to ensure that the heat exchange fluid in the heat exchange device 300 does not cause damage to the patient's internal tissue.

The devices and methods disclosed above may be used to perform temperature management in the entirety or a portion of an organism or a human body, to perform various therapies and to attain various purposes such as cell protection. The body temperature management devices and methods disclosed herein may be used by inserting such devices into, for example, the colon of a patient or an animal in order to perform temperature management of the person or animal.

Further and with reference to FIG. 10, a heat exchange device 400 may be particularly configured for the use of directly cooling or warming a patient's internal organs, such as their pancreas, liver, kidney, spleen, and intestines intraperitoneally during treatment of pancreatitis, liver and kidney infection or transplantation, liver, kidney, and intestine ischmia, and spleen enlargement. In this configuration, the heat exchange device 400 may include a variable cross-section pad 402 with a hard or soft surface, while in other embodiments, the embedded heat exchange device 400 may be in the same form as heat exchange device 300 discussed above. The embedded heat exchange device 400 is configured to perform heat exchange within or around a patient's organs, and is placed on the patient's organs to add or remove heat, as described above.

The embedded heat exchange device 400 may be formed of a temperature conductive material, such as steel, copper, or the like. Further, the embedded heat exchange device 400 may include temperature changeable materials for directly cooling or warming of internal organs for the treatment of, by way of non-limiting example, ischemia-reperfusion injury, trauma, and inflammation such as pancreatitis. The embedded heat exchange device 400 connects pad 402 to, for example, a regulator 406 and/or a temperature management source 408 and/or a control unit 200 (configured as discussed above), with such sections interconnecting via tubes 404 or electrically-conductive wires. The heat exchange medium used in embedded heat exchange device 400 may be a cooling or warming fluid, liquid or gas, such as liquid nitrogen, liquid argon, liquid oxygen, and their stream, or dry ice, and the like. Furthermore, embedded temperature management device 400 may include a refrigeration system and a Peltier device.

Guidewire 344, camera 220, light 174, steerable tip 350, and other features as described above may be assembled to embedded heat exchange device 400 for inserting the heat exchange device 400 inside or around internal organs such as the patient's pancreas, liver, intestines, and kidney.

While the exemplary embodiments have been shown and described, it will be understood by those skilled in the art that various changes in form and details may be made thereto without departing from the spirit and scope of the present disclosure as defined by the appended claims.

In addition, many modifications can be made to adapt a particular situation or material to the teachings of the present disclosure without departing from the essential scope thereof. Therefore, it is intended that the present disclosure not be limited to the particular exemplary embodiments disclosed as the best mode contemplated for carrying out the present disclosure, but that the present disclosure will include all embodiments falling within the scope of the appended claims.

What is claimed is:

1. A heat exchange device for the internal temperature management of a patient, comprising:
a heat transfer medium carrier having an expandable outer membrane, at least one heat exchange medium inlet channel, and at least one heat exchange medium outlet channel in fluid communication with said at least one heat exchange medium inlet channel; and
a guidewire lumen extending through said heat transfer medium carrier;
wherein said guidewire lumen is configured to house a guidewire to engage said heat transfer medium carrier to transfer movement from the guidewire to said heat transfer medium carrier.

2. The heat exchange device of claim 1, further comprising a plurality of heat exchange medium inlet channels and a plurality of heat exchange medium outlet channels.

3. The heat exchange device of claim 2, wherein said plurality of heat exchange medium inlet channels and said plurality of heat exchange medium outlet channels are arranged radially around said guidewire lumen.

4. The heat exchange device of claim 3, wherein each said heat exchange medium inlet channel is in fluid communication with one of said heat exchange medium inlet channels at a distal end of said heat transfer medium carrier.

5. The heat exchange device of claim 4, further comprising a fluid control unit, wherein said heat exchange medium inlet channels and said heat exchange medium outlet channels are in fluid communication with said fluid control unit to define a recirculating heat transfer fluid circuit.

6. The heat exchange device of claim 1, further comprising a guidewire in said guidewire lumen, said guidewire further comprising a stiffening structure that stiffens a portion of said guidewire to limit bending or compression of said guidewire in said portion.

7. The heat exchange device of claim 1, further comprising a guidewire in said guidewire lumen, said guidewire further comprising an adjustable stiffness mechanism configured to modify a stiffness of a portion of said guidewire.

8. The heat exchange device of claim 1, further comprising a steerable guidewire control apparatus configured to engage the guidewire and to change a direction of the guidewire in response to operation of said control apparatus.

9. The heat exchange device of claim 1, further comprising at least one inflatable balloon positioned about and surrounding at least a portion of said expandable outer membrane.

10. The heat exchange device of claim 9, further comprising a second inflatable balloon positioned about and surrounding at least a portion of said expandable outer membrane, wherein said first inflatable balloon is positioned adjacent a distal end of said heat transfer medium carrier, and said second inflatable balloon is positioned proximally to said first inflatable balloon.

11. The heat exchange device of claim 10, wherein said first inflatable balloon is moveable with respect to said expandable outer membrane.

12. The heat exchange device of claim 11, further comprising at least one fluid port on said heat transfer medium carrier and between said first inflatable balloon and said second inflatable balloon.

13. The heat exchange device of claim 1, further comprising a reinforcement structure surrounding a portion of said expandable outer membrane and expandable upon inflation of said expandable outer membrane.

14. The heat exchange device of claim 1, further comprising a temperature sensor within said heat transfer medium carrier.

15. The heat transfer device of claim 1, further comprising a pressure sensor within said heat transfer medium carrier.

16. The heat transfer device of claim 1, further comprising a flow sensor within said heat transfer medium carrier.

17. The heat transfer device of claim 1, further comprising an endoscopic camera attached to said heat transfer medium carrier.

18. The heat transfer device of claim 1, further comprising an irrigation lumen in said heat transfer medium carrier.

19. A heat exchange device for the internal temperature management of a patient, comprising:
a heat transfer medium carrier having an expandable outer membrane, at least one heat exchange medium inlet channel, and at least one heat exchange medium outlet channel in fluid communication with said at least one heat exchange medium inlet channel; and
at least one inflatable balloon positioned about and surrounding at least a portion of said expandable outer membrane.

20. The heat exchange device of claim 19, further comprising a guidewire lumen extending through said heat transfer medium carrier, wherein said guidewire lumen is configured to house a guidewire therein.

21. The heat exchange device of claim 19, further comprising a second inflatable balloon positioned about and surrounding at least a portion of said expandable outer membrane, wherein said first inflatable balloon is positioned adjacent a distal end of said heat transfer medium carrier, and said second inflatable balloon is positioned proximally to said first inflatable balloon.

22. The heat exchange device of claim 21, wherein said first inflatable balloon is moveable with respect to said expandable outer membrane.

23. The heat exchange device of claim 22, further comprising at least one fluid port on said heat transfer medium carrier and between said first inflatable balloon and said second inflatable balloon.

24. A method of managing a patient's temperature, comprising:
providing a heat exchange device for the internal temperature management of a patient, comprising:
a heat transfer medium carrier having an expandable outer membrane, at least one heat exchange medium inlet channel, and at least one heat exchange medium outlet channel in fluid communication with said at least one heat exchange medium inlet channel; and
a guidewire lumen extending through said heat transfer medium carrier;
wherein said guidewire lumen is configured to house a guidewire to engage said heat transfer medium carrier to transfer movement from the guidewire to said heat transfer medium carrier;
moving said heat exchange device through a portion of a patient's body through guidewire manipulation of said heat exchange device to a location of intended temperature management within said patient's body; and
causing said heat exchange device to affect heat transfer between the heat exchange device and the patient's tissue.

25. The method of claim 24, wherein said heat exchange device further comprises a first inflatable balloon moveably positioned about and surrounding at least a portion of said expandable outer membrane, and a second inflatable balloon positioned about and surrounding at least a portion of said expandable outer membrane, and at least one fluid port on said heat transfer medium carrier and between said first inflatable balloon and said second inflatable balloon, the method further comprising:
inflating said first inflatable balloon and said second inflatable balloon; and
flowing fluid through said fluid port to fill a space in said patient's body between said first inflatable balloon and said second inflatable balloon, and causing said first inflatable balloon to move toward a distal end of said heat transfer medium carrier.

26. The method of claim 25, further comprising the steps of:
evacuating said fluid from said space in said patient's body between said first inflatable balloon and said second inflatable balloon;
deflating said second inflatable balloon; and
causing said first inflatable balloon to pull said heat exchange device further into said patient's body.
